(19) United States
(12) Patent Application Publication
(10) Pub. No.: US 2010/0178663 A1
(43) Pub. Date: Jul. 15, 2010

(54) Apparatus And Methods For The Detection Of Plasma Metanephrines

(75) Inventors: Kendon Stuart Graham, Hopkinton, MS (US); Ido P. Kema, Netherlands (NL); Donald P. Cooper, Macclesfield (GB)

Correspondence Address: Waters Technologies Corporation, 34 MAPLE STREET - LG, MILFORD, MA, 01757, US

(73) Assignee: WATERS TECHNOLOGIES CORPORATION, Milford, MA (US)

(21) Appl. No.: 12/598,690

(22) PCT Filed: May 13, 2008
(86) PCT No.: PCT/US08/63510

§ 371 Date: Mar. 11, 2010

Related U.S. Application Data
(60) Provisional application No. 60/938,011, filed on May 15, 2007.

(51) Int. Cl. C12Q 1/02; C12M 1/34

(52) U.S. Cl. 435/29; 435/288.6

(57) ABSTRACT

Embodiments of the present invention feature the determination of normetanephrine, metanephrine and 3-methoxytyraminein raw human plasma with minimal sample pretreatment using a weak cationic extraction media to provide a selective sample clean-up and HILIC chemistries as indicators of pheochromoacytoma.

### STATEMENT REGARDING FEDERAL SPONSORSHIP

The present invention was made without Federal funds.

### FIELD OF THE INVENTION

Embodiments of the present invention are directed to the detection of metanephrines in blood plasma for the diagnosis of pheochromocytoma.

### BACKGROUND OF THE INVENTION

Pheochromocytoma is tumor of the adrenal medulla. These tumors produce catecholamines which are released into the blood stream. In the blood stream, the catecholamines cause individuals so inflicted to experience hypertension. The hypertension may present itself as a chronic condition or episodic corresponding to the manner in which the tumor or tumors release the catecholamines. Although relatively rare, it is desirable to diagnose the presence or absence of pheochromocytoma before treating hypertensive patients.

The methods of detecting pheochromocytoma have been awkward and time consuming. The methods have also been plagued by false positive and false negative results.

### SUMMARY OF THE INVENTION

Embodiments of the present invention are directed to methods and apparatus for determining levels of normetanephrine, metanephrine and 3-methoxytyramine in blood plasma. The terms “normetanephrine”, “metanephrine”, and “3-methoxytyramine” are used in the ordinary chemical sense as such compounds are known in the art. The term “blood plasma” is also used as such term is known in the art. Normetanephrine, metanephrine and 3-methoxytyramine are catecholamine metabolites. This paper will use the term “3-methoxytyramine to refer to such compound. The presence and abnormal concentration of normetanephrine, metanephrine and 3-methoxytyramine in plasma is potentially indicative of catecholamine secreting tumors.

Embodiments of the present invention directed to a method comprising the steps of obtaining a plasma sample from a subject and separating normetanephrine, metanephrine and 3-methoxytyramine, if present, from plasma proteins and constituents with a weak cationic exchange resin to form at least one, if present, retained normetanephrine, metanephrine and 3-methoxytyramine. The retained normetanephrine, metanephrine and 3-methoxytyramine are eluted from the weak cationic exchange resin with an acid mobile phase to form at least one normetanephrine, metanephrine and 3-methoxytyramine eluant. The normetanephrine, metanephrine and 3-methoxytyramine eluant is separated into at least one fine normetanephrine, metanephrine and 3-methoxytyramine eluant through on line hydrophilic interaction liquid chromatography. The fine normetanephrine, metanephrine and 3-methoxytyramine eluant exhibiting one or more peaks corresponding to the presence of at least one of group consisting of normetanephrine, metanephrine and 3-methoxytyramine is introduced into a mass spectrometer to produce a mass to charge signal relating to the presence of and quantity of at least one of the group consisting of normetanephrine, metanephrine and 3-methoxytyramine. The presence and abnormal quantity of at least one of the group of normetanephrine, metanephrine and 3-methoxytyramine is indicative of the presence or absence of tumors secreting catecholamines.

Preferably, the weak cationic exchange resin is a water-wettable organic polymer with one or more weak cationic exchange functional groups. A preferred organic polymer is derived from the monomers selected from at least one hydrophilic monomer and at least one hydrophobic monomer. By way of example without limitation, one preferred hydrophobic monomer is divinylbenzene. Preferred hydrophilic monomers include N-vinylpyrrolidone and vinylpyridine. One preferred weak cationic exchange group is carboxyl.

Preferably, the method further comprises the step of adding an internal standard to the sample. The internal standard is, preferably, at least one known quantity of a deuterated form of at least one of the group comprising normetanephrine, metanephrine and 3-methoxytyramine. Preferably, plasma levels of catecholamine metabolites are determined by comparing unlabeled normetanephrine levels, unlabeled metanephrine levels, and unlabeled 3-methoxytyramine levels to deuterated normetanephrine, deuterated metanephrine and deuterated 3-methoxytyramine concentrations in the sample.

Preferably, the mass spectrometer is operated in positive ionization mode. Metanephrine is protonated to form an ion of mass to charge 198. Normetanephrine is protonated to form an ion of mass to charge 184 3-methoxytyramine is protonated to form an ion of mass to charge ration of 168. Metanephrine will typically lose water to form an ion of having a mass to charge of 180. Normetanephrine will typically lose water to form an ion of having a mass to charge of 166. 3-methoxytyramine will typically lose ammonia to form an ion having a mass to charge ratio of 151.

Preferably, the mass spectrometer employs collisional fragmentation to form one or more ion fragments of metanephrine having a mass to charge of 148, one or more ion fragments of normetanephrine having a mass to charge of 134, and one or more ion fragments of 3-methoxytyramine having a mass to charge ratio of 119.

The presence of the fragments or whole molecules and the concentration as determined by the mass spectrometer is related to the sample. These concentrations are compared control values. A concentration of metanephrine greater than about 0.3 nmole per liter suggests pheochromocytoma. A concentration of normetanephrine greater than about 1.2 nmole per liter suggests pheochromocytoma. A concentration of 3-methoxytyramine greater than about 0.2 nmole per liter suggests pheochromocytoma.

A further embodiment of the present invention is directed to an apparatus for detecting normetanephrine, metanephrine and 3-methoxytyramine in blood plasma. Again, the presence and abnormal concentration of normetanephrine, metanephrine and 3-methoxytyramine is potentially indicative of catecholamine secreting tumors. The apparatus comprises separating and eluting means for separating at least one of the group normetanephrine, metanephrine and 3-methoxytyramines from plasma proteins and constituents with a weak cationic exchange resin to form at least one of the group of retained normetanephrine, metanephrine and 3-methoxytyramine, and eluting the retained normetanephrine, metanephrine and 3-methoxytyramine from the weak cationic exchange resin with an acid mobile phase to form a at least one of the group of normetanephrine, metanephrine and 3-methoxytyramine eluant. The separating and eluting means receives one or more plasma samples from one or more subjects, and is in fluid communication with liquid chromatograph means. The separating and eluting means is in signal communication with control means to send a sample identifier signal. The apparatus further comprises liquid chromatograph means for separating the at least one of the group of normetanephrine, metanephrine and 3-methoxytyramine eluant into at least one of the group comprising a fine normetanephrine, metanephrine and 3-methoxytyramine eluant through on line hydrophilic interaction liquid chromatography. The fine normetanephrine, metanephrine and 3-methoxytyramine eluant exhibits one or more peaks corresponding to the presence of at least one of group consisting of metanephrine, normetanephrine and 3-methoxytyramine. The liquid chromatograph means is in fluid communication with mass spectrometer means and is in signal communication with control means. The liquid chromatograph maintains the fine normetanephrine, metanephrine and 3-methoxytyramine eluant in association with the sample identifier signal. The apparatus further comprises mass spectrometer means for receiving the at least one of fine normetanphrine, metanephrine and 3-methoxytyramine eluant to produce at least one mass to charge signal relating to the presence of and quantity of at least one of the group consisting of normetanephrine, metanephrine and 3-methoxytyramine. The mass spectrometer means is in signal communication with control means. The apparatus further comprises control means for receiving the sample identifier signal and mass to charge signal and associating the mass to charge signal to the sample identifier signal. The control means compares the mass to charge signal to control values to suggest the presence or absence of tumors secreting catecholamines to a subject.

Separating and eluting means, in the context of the present invention, refers to sample preparative systems used to separate compounds having grossly different retention characteristics. Liquid chromatograph means refers to separation systems capable of separations at an analytical level. Mass spectrometer means refers to any form of mass spectrometer such as ion trap, single mass spectrometers, double and triple quadrupole mass spectrometers, and time of flight (TOF) mass spectrometers, by way of example. As used herein, the term “fluid communication” refers to plumbed to receive or transfer fluids and encompasses the injection of fluid into or from. The term in “signal communication refers to transmission of signals, to receive or transmit data or instructions or commands in an electromagnetic, optical manner, by wire, optical fiber or wireless radio communications.

A further embodiment of the present invention is directed to a kit for performing analysis of at least one of the group metabolites of naturally occurring catecholamines in plasma selected from metanephrine, normetanephrine and 3-methoxytyramine. The kit comprises standards and calibrators for a mass spectrometer for performing an analysis with suitable instructions for their use. The kit preferably has suitable packaging and enclosures for the reagents and materials provided.

These and other features and advantages will be apparent upon viewing the figures and reading the detailed description that follow.

### BRIEF DESCRIPTION OF THE DRAWINGS

FIG. 1 depicts a schematic of an instrument embodying features of the present invention;

FIG. 2 depicts a kit embodying features of the present invention;

FIG. 3a depicts a product ion spectra of normetanephrine obtained by mass spectrometry;

FIG. 3b depicts a product ion spectra of metanephrine obtained by mass spectrometry;

FIG. 4a depicts a calibration line obtained by injecting plasma samples spiked with metanephrine;

FIG. 4b depicts a calibration line obtained by injecting plasma samples spiked with normetanephrine;

FIGS. 5a and 5b depict multiple reaction monitoring chromatograms for plasma samples containing 0.16 nmole/L metanephrine and a approximately 1 nmole/L of d3-metanephrine;

FIGS. 5c and 5d depict multiple reaction monitoring chromatograms for plasma samples containing 0.38 nmol/L normetanephrine and a approximately 1 nmole/L of 3-normetanephrine;

FIG. 6a depicts reference ranges for metanephrine from a small set (n=102) of plasma samples obtained from patients assumed to be healthy; and,

FIG. 6b depicts reference ranges for normetanephrine from a small set (n=102) of plasma samples obtained from patients assumed to be healthy; and,

FIG. 7 depicts chromatograms of free plasma metanephrine, normetanephrine and 3-methoxytyramine and their deuterated internal standards by liquid chromatography and MS/MS in MRM mode.

FIG. 8a depicts distributions of fractionated plasma free metanephrine in reference samples from 120 individuals in nmoles per liter.

FIG. 8b depicts distributions of fractionated plasma free normetanephrine in reference samples from 120 individuals in nmoles per liter.

FIG. 8c depicts distributions of fractionated plasma free 3-methoxytyramine in reference samples from 120 individuals in nmoles per liter.

### DETAILED DESCRIPTION OF THE INVENTION

Embodiment of the present invention will be described with respect to an methods and apparatus for detecting at least one of the group of compounds selected from normetanephrine, metanephrine and 3-methoxytyramine in blood plasma and a kit for the detection of at least one of the group of compounds selected from normetanephrine, metanephrine and 3-methoxytyramine in blood plasma. The apparatus, kits and methods have utility for the detection of the presence and abnormal concentration of at least one of the group of compounds selected from normetanephrine, metanephrine and 3-methoxytyramine in plasma samples. Abnormal elevations of normetanephrine, metanephrine and 3-methoxytyramine in plasma are potentially indicative of catecholamine secreting tumors. Those skilled in the art will recognize the diagnostic utility of embodiments of the present invention for the diagnosis of pheochromocytoma and other disease states involving catecholamine secreting tumors.

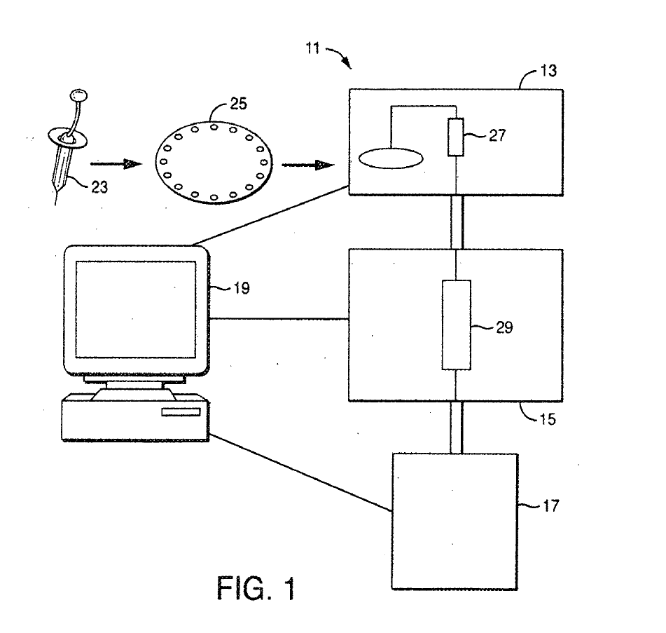

An apparatus for the detection of at least one of the group of compounds selected from normetanephrine, metanephrine and 3-methoxytyramine in blood plasma, generally designated by the numeral 11, is depicted in FIG. 1. The apparatus comprises the following major elements: separating and eluting means 13, liquid chromatograph means 15, mass spectrometer means 17 and control means 19.

Separating and eluting means 13 is for separating, each from the other and each, normetanephrine, metanephrine and 3-methoxytyramine, from plasma proteins and constituents. The separation and eluting means receives one or more samples obtained from patients, as depicted schematically by the syringe 23. The samples are obtained as blood samples and processed by centrifugation to separate blood cells from the plasma.

Preferably, the samples are diluted with a standard dilution solution containing a known quantity of labeled normetanephrine, labeled metanephrine and labeled 3-methoxytyramine. A preferred labeled normetanephrine, metanephrine and 3-methoxytyramine are deuterated normetanephrine, deuterated metanephrine and deuterated 3-methoxytyramine which will serve as internal standards. The labeled normetanephrine, labeled metanephrine and labeled 3-methoxytyramine allow the quantification of the unlabeled normetanephrine, unlabeled metanephrine and unlabeled 3-methoxytyramine by mass spectrometer means 17.

The diluted plasma samples are loaded into vials, depicted as nesting in a circular tray 25, or extraction cartridges [not shown] or trays of multiwell plates [not shown]. Extraction cartridges and multiwell plates are well known in the art. The samples are indexed so that the vials, or wells or tray positions can be identified to a patient by optical or electronic means such as bar coding, magnetic strips or memory chips and entered into the control means 19.

The plasma is forced through a weak cationic exchange resin in the separating and eluting means to form at least one retained normetanephrine, metanephrine and 3-methoxytyramine, if present. The weak cationic exchange resin is held in the cartridge column, vial or multiwell plate or in a separate column within the separating and eluting means. As depicted, the vials are received in the separating and eluting means 13 and aliquots are drawn and directed into a column 27.

The column 27, cartridge column, vial, or multiwell plate is packed with a weak cationic exchange resin. Preferably, the weak cationic exchange resin is water wettable allowing the media to be wetted and rewetted. A preferred resin is a water wettable weak cationic exchange resin having an organic polymeric composition. A preferred organic polymer is derived from the monomers selected from at least one hydrophilic monomer and at least one hydrophobic monomer. By way of example without limitation, one preferred hydrophobic monomer is divinylbenzene. Preferred hydrophilic monomers include N-vinylpyrrolidone and vinylpyridine. One preferred weak cationic exchange group is carboxyl. A preferred polymeric composition is sold under the trademark OASIS® WCX (Waters Corporation, Milford, Mass., USA)

Separating and eluting means 13 is preferably an on-line solid phase extraction system for sample processing. These systems are available from several vendors such as the Symbiosis® Pharma System sold by Spark Holland (Emmen, The Netherlands). The separating and eluting means 13 is in signal communication with control means 19 and in fluid communication liquid chromatograph means 15.

Control means 19 receives the indexing data from the separating and eluting means 13 to allow data from the down stream components to relate back to individual subjects from where the sample was derived. Control means 19 is a computer type device such as an computer processing unit (CPU) integrated into the device 11, or computer, including by way of example without limitation, a mainframe computer, portable, desktop, laptop or server type computer with appropriate software controls. Computers are sold by a large number of vendors such as Apple Computers, Inc, of Cupertino, Calif., USA or Dell Corporation of Houston, Tex., USA. A preferred software system comprises Sparklink v.3.0 available from Spark Holland and MassLynx v.4.0 available from Waters Corporation.

Separating and eluting means is in fluid communication with a source of an acid mobile phase [not shown]. At least one of normetanephrine, metanephrine and 3-methoxytyramine, if present, are retained on the column 27, and other plasma constituents are directed to waste [not shown]. Control means directs the separation and eluting means 13 to pump an acid mobile phase through the column 27 to form at least one of a normetanephrine, metanephrine and 3-methoxytyramine eluant, if such compound was originally present in the sample.

The normetanephrine, metanephrine and/or 3-methoxytyramine eluant is directed into liquid chromatograph means 15. Liquid chromatograph means 15 is for separating each normetanephrine, metanephrine and 3-methoxytyramine eluant into at least one fine normetanephrine eluant, fine metanephrine eluant and fine 3-methoxytyramine eluant through on line hydrophilic interaction liquid chromatography. Liquid chromatograph means 15 are available from several vendors such as the ALLIANCE® separations module and the ACQUITY® separations module available from Waters Corporation and 1100 Chromatography System available from Agilent Corporation (Palo Alto, Calif., USA).

As used herein, the term “on-line” means that the separation is performed by high pressure chromatography. Hydrophilic interaction chromatography is a separation technique in which compounds are separated by passing a hydrophobic or mostly organic mobile phase across a neutral hydrophilic stationary phase causing compounds in solution to elute in order of increasing hydrophilicity.

Liquid chromatography means 15 has a fine column 29 to perform hydrophilic interaction chromatography. Fine column 29 is available from several venders, a preferred column 29 is an ATLANTIS® Brand column available from Waters Corporation packed with a silica particles. A preferred column has 3 micron particles and is 2.1 mm×50 mm. However, columns with different particle size and column dimensions can be readily substituted.

Each fine normetanephrine eluant, fine metanephrine eluant and fine 3-methoxytyramine eluant exhibits one or more peaks corresponding to the presence of at least one of group consisting of metanephrine, normetanephrine and 3-methoxytyramine. The liquid chromatograph means 15 is in fluid communication with mass spectrometer means 17 and is in signal communication with control means 19. The liquid chromatograph means 17 maintains each fine normetanephrine eluant, fine metanephrine eluant and fine 3-methoxytyramine eluant in association with the sample identifier signal maintained as data by the control means 19.

Mass spectrometer means 17 receives the fine normetanphrine, fine metanephrine eluant and/or fine 3-methoxytyramine eluant to produce at least one mass to charge signal relating to the presence of and quantity of at least one of said group consisting of normetanephrine, metanephrine and 3-methoxytyramine. The mass spectrometer means 17 is in signal communication with control means 19. Mass spectrometer means 17 is any form of mass spectrometer such as ion trap, single mass spectrometers, double and triple quadrupole mass spectrometers, time of flight (TOF) mass spectrometers by way of example. A preferred mass spectrometer means 17 is a QUATTRO MICRO® tandem mass spectrometer sold by Waters Corporation.

Control means 19 receives the sample identifier signal and mass to charge signal and associates the mass to charge signal to said sample identifier signal. The control means compares the mass to charge signal to control values to suggest the presence or absence of tumors secreting catecholamines to a subject and records and displays the results via a screen display, printout, or some other optical or sound transmission to the operator.

Preferably, the separation and eluting means 13 receives an internal standard which will be processed and directed through the liquid chromatograph means 15 and mass spectrometer means 17 prior to or simultaneously with the analysis to facilitate identification and quantification by the apparatus 11. The internal standard is, preferably, at least one known quantity of a deuterated form of normetanephrine, metanephrine and/or 3-methoxytyramine.

Preferably, plasma levels of catecholamines are determined by comparing unlabeled normetanephrine levels, unlabeled metanephrine levels and unlabeled 3-methoxytyramine levels to corresponding deuterated normetanephrine, deuterated metanephrine and deuterated 3-methoxytyramine concentrations in the sample.

Preferably, the mass spectrometer is operated in positive ionization mode. Metanephrine is protonated to form an ion of mass to charge 198. Normetanephrine is protonated to form an ion of mass to charge 184 3-methoxytyramine is protonated to form an ion of mass to charge ration of 168. Metanephrine will typically lose water to form an ion of having a mass to charge of 180. Normetanephrine will typically lose water to form an ion of having a mass to charge of 166. 3-methoxytyramine will typically lose ammonia to form an ion having a mass to charge ratio of 151.

Preferably, the mass spectrometer employs collisional fragmentation to form one or more ion fragments of metanephrine having a mass to charge of 148, and one or more ion fragments of normetanephrine having a mass to charge of 134, and one or more ion fragments of 3-methoxytyramine having a mass to charge ratio of 119.

The presence of the fragments or whole molecules and the concentration as determined by the mass spectrometer is related to the sample. These concentrations are compared control values by control means 19 or by the operator. A concentration of metanephrine greater than about 0.3 nmole per liter suggests pheochromocytoma. A concentration of normetanephrine greater than about 1.2 nmole per liter suggest pheochromocytoma. A concentration of 3-methoxytyramine greater than about 0.2 nmole per liter suggests pheochromocytoma.

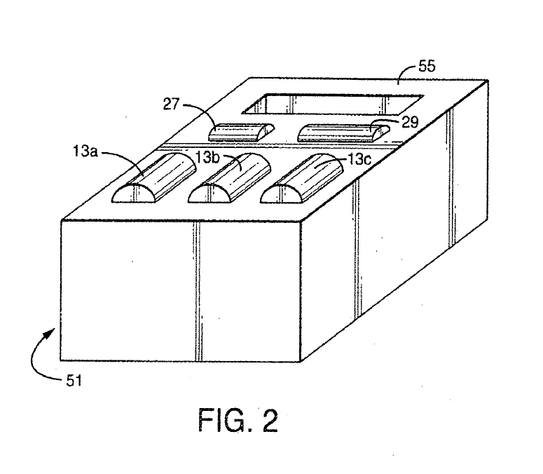

Turning now to FIG. 2, a kit for performing analysis of at least one of the group metabolites of naturally occurring catecholamines in plasma selected from metanephrine normetanephrine, and 3-methoxytyramine generally designated by the numeral 51, is depicted. The kit comprises internal standards and reagents for an apparatus 11.

The kit 51 has one or more vials or containers 13a, 13b and 13c containing metanephrine, normetanephrine, respectively. The compounds will be used to form calibration solutions containing known amounts of normetanephrine, metanephrine and 3-methoxytyramine in labeled and unlabeled forms. The kit 51, preferably, has a column 27 and a fine column 29. Preferably, the kit 51 has instructions for performing an analysis. The kit preferably has suitable packaging, such as box 55.

Embodiments of the present invention directed to method will be described with respect to the following Examples.

### Example 1

### Patient Samples

Plasma samples from 6 healthy volunteers were provided by Medeval Laboratories (Manchester, UK) and were used to assess the performance characteristics of the assay and to prepare calibrators. A further 102 plasma samples were used in the preliminary investigation of reference ranges for metanephrine and normetanephrine. These were collected from patients assumed to be healthy and were provided by UMC Groningen (Groningen, The Netherlands).

### Standards, Calibrators & Quality Controls

Normetanephrine and metanephrine were purchased from Sigma Aldrich Ltd (Poole, UK) as D,L-metanephrine.HCl and D,L-normetanephrine.HCl. The deuterated internal standards α,α,β-d3-metanephrine.HCl and α,α,β-d3-normetanephrine.HCl were purchased from Cambridge Isotopes Inc. (Andover, Mass., USA) and Medical Isotopes Inc. (Pelham, N.H., USA), respectively.

Calibrators were prepared by spiking 1 mL plasma samples with normetanephrine and metanephrine (10 μL) made up in 0.1M HCl prior to thorough mixing. QC samples were prepared in a similar manner using stock solutions of M and NM that were independent of those used to prepare the calibrators.

### Mass Spectrometry

A Quattro micro tandem mass spectrometer with a Z SPRAY ion source was used for all analyses (Waters Corporation, Manchester, UK). This instrument was operated in positive ionisation mode and was coupled directly to a Symbiosis® Pharma (Spark Holland, Emmen, The Netherlands) on-line solid phase extraction—liquid chromatography system. MS System control and data acquisition was performed using MassLynx v4.0 software with automated data processing by the QuanLynx Application Manager. Control of the Symbiosis system was performed using SparkLink v3.0 software.

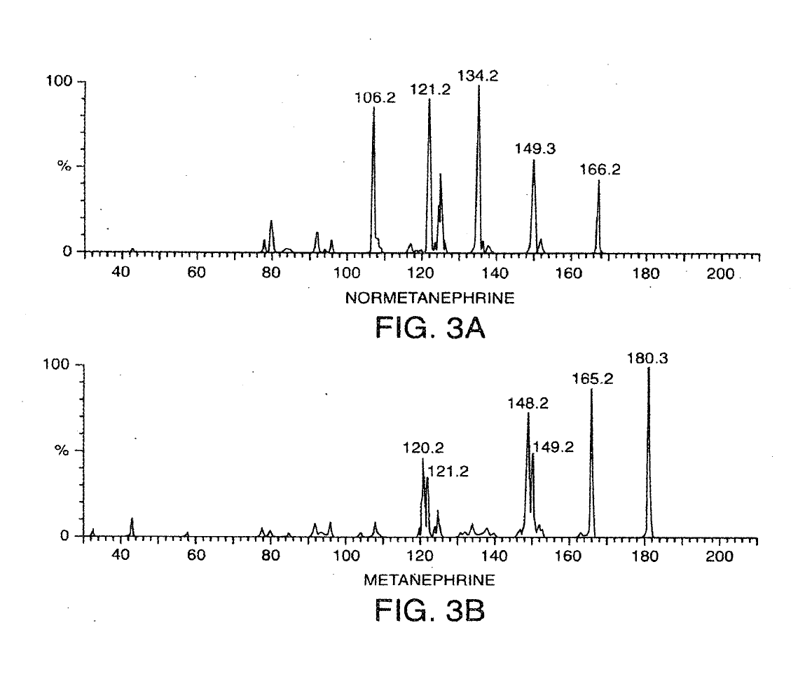

In positive ionization mode, normetanephrine and metanephrine are protonated to produce ions of the form [M+H]⁺ of m/z 198 and m/z 184, respectively. These ions are known to then undergo a facile loss of water¹⁰ and the ion source conditions were optimised for these resulting ions (M=m/z 180; NM=m/z 166) of the form [M+H—H₂O]⁺. Upon collision induced dissociation (CID), these precursor ions produced characteristic product ions of m/z 148 and m/z 134 for M and NM metanephrine and normetanephrine, respectively, as best seen in FIGS. 3a and 3b.

Using the information from these experiments, the MS Method shown in table 1 was used to monitor normetanephrine and metanephrine and their deuterated analogues in MRM mode using a dwell time of 0.07 sec.

TABLE 1

| | | | | Collision |
|---|---|---|---|---|
| | Precursor | Product | Cone | Energy |
| Compound | (m/z) | (m/z) | Voltage (V) | (eV) |
| Metanephrine | 180.1 | 148.1 | 20 | 17 |
| D3-Metanephrine | 183.1 | 151.1 | 20 | 17 |
| Normetanephrine | 166.1 | 134.1 | 18 | 16 |
| D3-Normetanephrine | 169.1 | 137.1 | 18 | 16 |

ON-LINE SOLID PHASE EXTRACTION

| Sample Volume: | 40 μL (1:1 dilution of plasma with aqueous | |
|---|---|---|
| | IS solution) | |
| Cartridge: | Waters 10 mm × 1 mm Oasis ® WCX | |
| Solvation: | 1 mL Acetonitrile | 5 mL/min |
| Equilibration: | 1 mL Water | 5 mL/min |
| Sample Loading: | 1 mL Water | 2 mL/min |
| Wash 1: | 1 mL Water | 5 mL/min |
| Wash 2: | 1 mL Acetonitrile | 5 mL/min |

| | |
|---|---|
| Elution Duration: | 2 minutes with LC Mobile Phase |
| Total Extraction Time: | 2 min 55 sec including Valve Wash |
| Total Cycle Time: | 7 min 40 sec per sample |

LIQUID CHROMATOGRAPHY CONDITIONS

| | |
|---|---|
| Column: | Waters 2.1 mm × 50 mm HILIC; 3 μm |
| Mobile Phase A: | Acetonitrile |
| Mobile Phase B: | 100 mM Ammonium Formate @ pH 3 |

| Time (m:ss) | Flow (mL/min) | % A | % B |
|---|---|---|---|
| 0:00 | 0.3 | 95 | 5 |
| 0:05 | 0.3 | 95 | 5 |
| 4:10 | 0.3 | 80 | 20 |
| 4:40 | 0.3 | 80 | 20 |
| 4:41 | 0.3 | 95 | 5 |
| 7:15 | 0.3 | 95 | 5 |

### Results

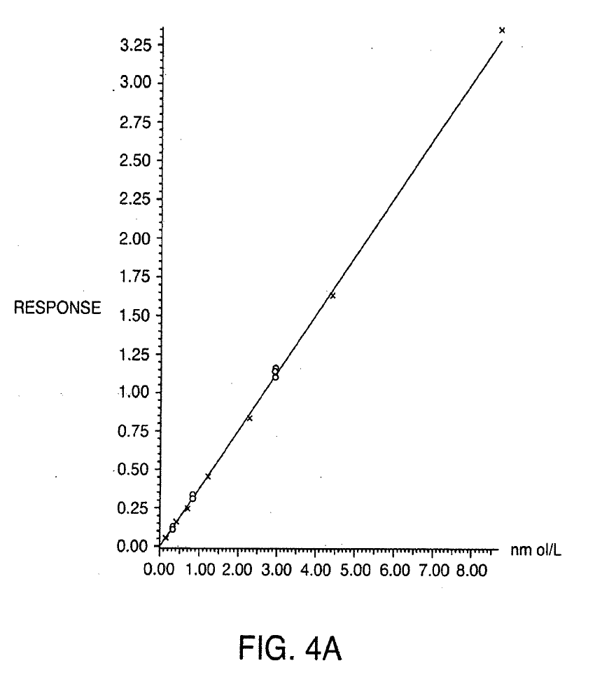

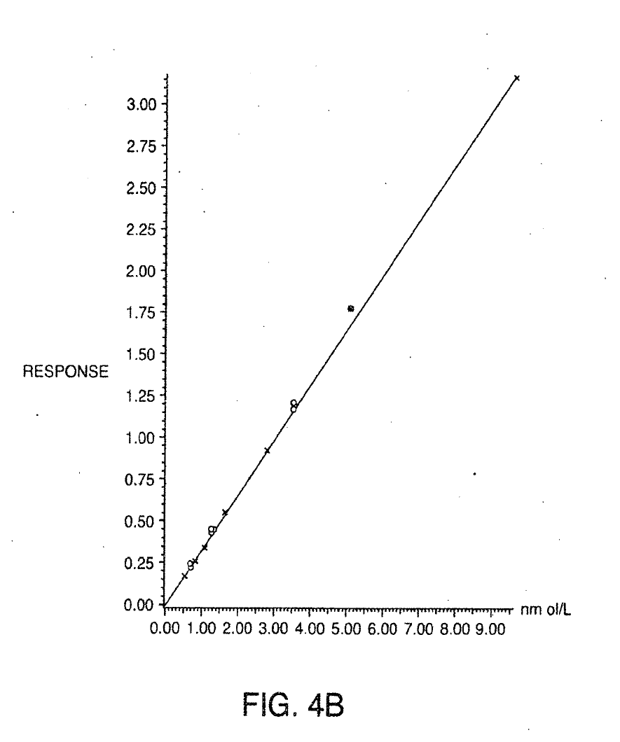

The calibration lines were linear over the examined range with correlation co-efficients >0.999 for normetanephrine and metanephrine as best seen in FIGS. 4a and 4b.

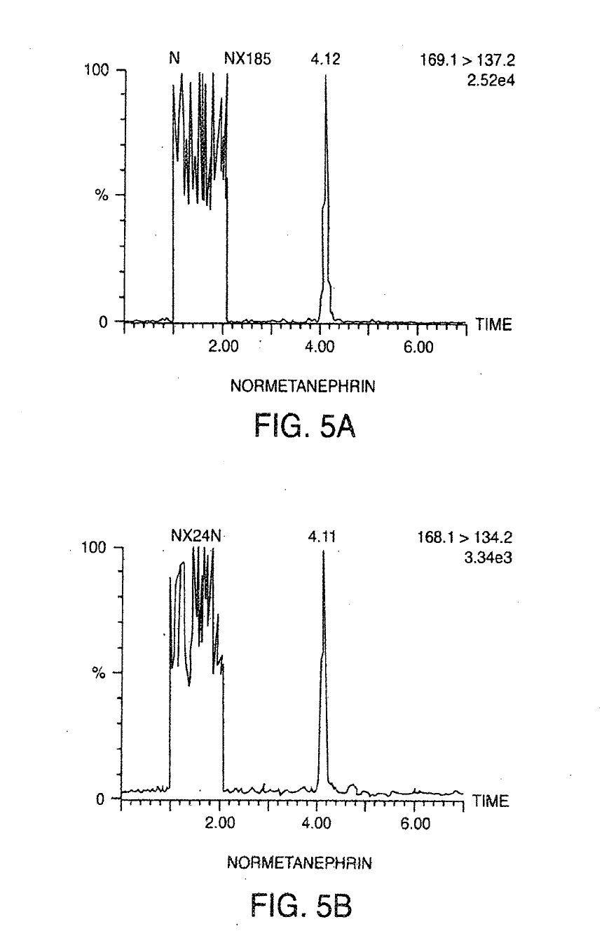

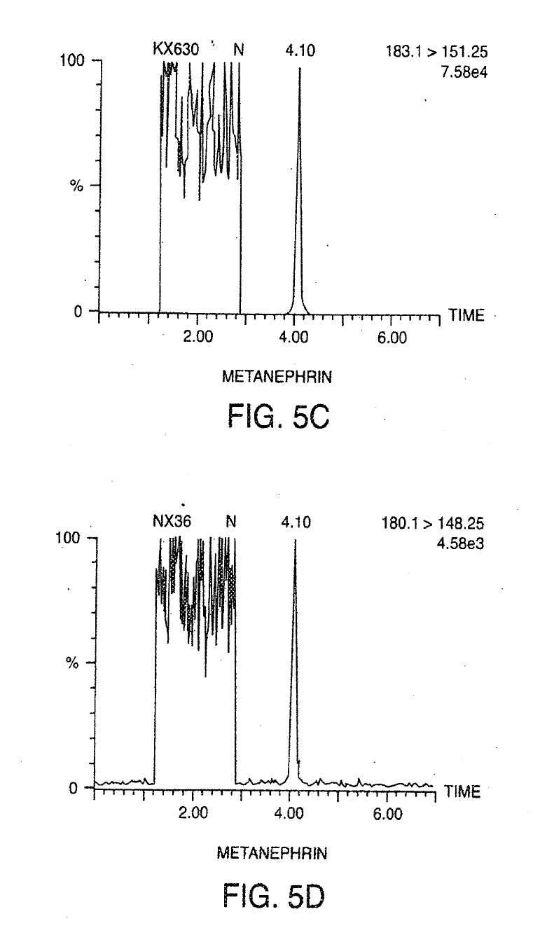

The lower limit of quantification (signal-to-noise ratio ≧10) for M and NM were 0.04 and 0.16 nmol/L, respectively as best seen in FIGS. 5a, 5b, 5c and 5d.

Extraction recoveries for M and NM were found to be ≧90% using the automated Method Development function of the Symbiosis® Pharma system.

Intra-assay variation was calculated using QC samples at three levels for M and NM. This was found to be <6% at all levels (Table 2).

TABLE 2

| | Metanephrine | | Normetanephrine | |
|---|---|---|---|---|
| | Mean ± Std Dev | | Mean ± Std Dev | |
| Patient | (nmol/L) | % CV | (nmol/L) | % CV |
| QC Level 1 | 0.29 ± 0.02 | 5.3 | 0.76 ± 0.04 | 5.8 |
| QC Level 2 | 0.82 ± 0.03 | 3.2 | 1.57 ± 0.06 | 3.8 |
| QC Level 3 | 2.93 ± 0.07 | 2.3 | 3.56 ± 0.09 | 2.6 |

Inter-assay variation was calculated using QC samples at three levels (n=10) and the results from patient samples over several assays (n=7). In both cases, inter-assay precision was found to be ≦15% (Table 3).

TABLE 3

| | Metanephrine | | Normetanephrine | |
|---|---|---|---|---|
| | Mean ± Std Dev | | Mean ± Std Dev | |
| Patient | (nmol/L) | % CV | (nmol/L) | % CV |
| Patient 1 | 0.14 ± 0.02 | 12 | 0.57 ± 0.06 | 10 |
| Patient 2 | 0.16 ± 0.01 | 8.0 | 0.41 ± 0.06 | 15 |
| Patient 3 | 0.10 ± 0.02 | 15 | 0.40 ± 0.03 | 7.4 |
| Patient 4 | 0.14 ± 0.01 | 9.0 | 0.32 ± 0.05 | 14 |
| Patient 5 | 0.14 ± 0.02 | 14 | 0.55 ± 0.08 | 15 |
| Patient 6 | 0.17 ± 0.02 | 10 | 0.41 ± 0.06 | 15 |
| (n-7) | | | | |

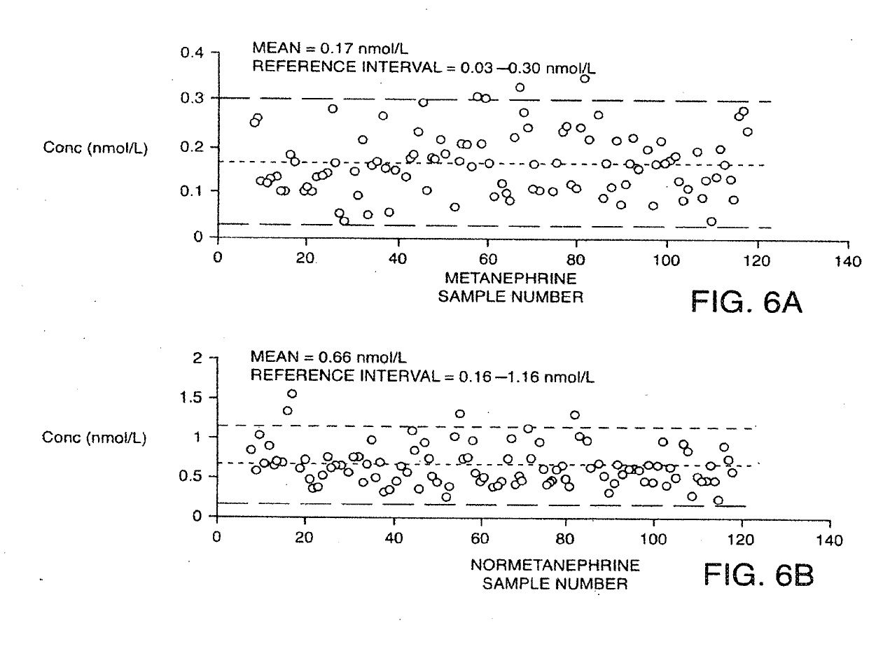

Provisional estimation of reference intervals for M and NM normetanephrine and metanephrine was based on the analysis of 102 patient samples who were assumed to be healthy. The reference intervals were calculated using the mean concentrations of M and NM normetanephrine and metanephrine found in the patient samples ±2 standard deviations as best seen in FIGS. 6a and 6b.

The use of on-line solid phase extraction technology coupled to LC-MS/MS has been shown to provide a PFM assay with improved sensitivity, selectivity and vastly reduced sample handling. Simple dilution of plasma samples with water containing deuterated internal standards followed by centrifugation now replaces tedious off-line extraction methods.

A highly-selective extraction process is achieved using weak cation exchange (WCX) media. Traditionally, strong bases are extracted using strong cation exchange (SCX) media where the base must be eluted via neutralisation. In the case of quarternary amines, this is often not possible and, more commonly, the stabilities of the basic analytes are compromised. Using the Waters Oasis™ WCX media, strong bases bind to the carboxyl ion-exchanger in the cartridge at pH >5 permitting the cartridge to be washed with water and 100% acetonitrile without elution of the analytes of interest. Elution of the cartridge is then carried out by allowing the acidic mobile phase used in the chromatographic method.

The use of HILIC Chemistry for the analysis of polar bases provides LC-MS/MS assays with higher sensitivities than traditional reversed-phase methods when using electrospray ionisation. The analytes of interest elute in high concentrations (circa 75%) of organic solvent where the desolvation process is more efficient.

As a preliminary indication of the validity of the assay, the M and NM normetanephrine and metanephrine levels in the small group of patient samples (n=102) was used to calculate tentative reference intervals (FIGS. 6a and 6b). These were found to be in close agreement with those in a previous study¹⁰ that suggests reference intervals of 0.05-0.47 nmol/L and 0.12-1.1 nmol/L for M and NM, respectively. It should be noted that specimen collection strategies may have important consequences on the M and NM normetanephrine and metanephrine levels, particularly the position of the patient when blood samples are obtained.

Determination of normetanephrine and metanephrine in raw human plasma with minimal sample pretreatment using the SPARK SYMBIOSIS® pharma system and the waters QUATTRO MICRO™ was demonstrated.the use of Water Oasis™ WCX extraction media can be used to provide a selective sample clean-up for highly basic analytes in a complex matrix.the WATERS QUATTRO MICRO™ used in conjunction with waters HILIC chemistries can provide a sensitive method of analysis for low levels of highly polar, basic analytes.

### Example 2

This example uses the abbreviations “MN” for metanephrine, “NMN” for normetanephrine and “3-MT” for 3-methoxytyramine.

### Materials and Methods

### Reagents

HPLC-grade acetonitrile and methanol were obtained from Rathburn Chemicals Ltd (Walkerburn, Scotland); ammonium phosphate 99.995⁺% and disodium EDTA were purchased from Sigma-Aldrich Ltd (Steinheim, Germany). Formic acid 98-100% ultra-pure was supplied by BDH Laboratory Supplies (Poole, UK); ortho-phosphoric acid 95% (p.a.), 2-propanol (p.a.) and sodium metabisulfite (Na₂S₂O₅) were all purchased from Merck KGaA (Darmstadt, Germany). Sodium hydroxide (NaOH) and hydrochloric acid were homemade. Reagent-grade water, obtained from a Barnstead system, was used throughout.

D,L-metanephrine.HCl, D,L-normetanephrine.HCl and D,L-3-methoxytyramine.HCl were purchased from Sigma-Aldrich Ltd (Poole, UK). The deuterated internal standards α,α,β-d3-metanephrine.HCl and α,α,β,β-d4-3-methoxytyramine.HCl were purchased from Cambridge Isotopes Inc. (Andover, Mass.); α,α,β-d3-normetanephrine.HCl was purchased from Medical Isotopes Inc. (Pelham, N.H.).

### Stock Solutions and Samples

Stock solutions were prepared in 0.1 mol/L HCl. Stock solutions were serially diluted and used to form calibrators and low, medium and high quality control samples in pooled plasma via spiking. The concentration range of the calibrators was from physiological levels (0-1 nmol/L) to approximately 20 nmol/L for all analytes.

Plasma samples from healthy controls and patients with confirmed pheochromocytoma (as established by the routinely used GC-MS method for urinary fractionated metanephrines and pathology reports) came from the University Medical Center, University of Grotingen, Groningen, The Netherlands (UMCG). Blood samples were collected by venepuncture, with the patient in sitting position, in 10 mL Vacutainer Tubes (Becton and Dickinson, Franklin Lakes, N.J.) containing K₂EDTA solution as anticoagulant. After centrifugation, the resultant plasma was transferred to glass tubes containing 150 mg Na₂S₂O₅ as a preservative. Samples were stored at −20° C. until analysis.

Prior to analysis, aliquots of plasma samples (500 μL) were mixed with 100 μL internal standard stock solution (4.95 nmol/L in diluted acid) and diluted with 400 μL water. Sample vials were placed in the autosampler and 100 μL of each sample (equivalent to 50 μL of plasma) was injected.

### Analysis and Quantification

Instrumentation. A Spark Holland Symbiosis® (Spark Holland, Emmen, The Netherlands) on-line solid-phase extraction system was used for all analysis. The system consists of a temperature-controlled autosampler (temperature maintained at 10° C.), a SPE controller unit (automated cartridge exchanger; ACE), a solvent delivery unit (two high-pressure dispensers) and an HPLC pump. The ACE module contained two connectable 6-way valves and a SPE cartridge-exchange module. The high pressure dispensers provide SPE cartridges with solvents for conditioning, equilibration, sample application, and clean-up. The integrated HPLC pump used was a binary high-pressure gradient pump.

Oasis® WCX (weak cation exchange) 10 mm×1 mm SPE cartridges (Waters Corporation, Milford, Mass.) were used for sample extraction. HPLC was performed using an Atlantis HILIC Silica column (particle size 3 μm; 2.1 mm I.D.×50 mm; Waters). The temperature of the column was controlled with a Mistral Column Oven (Spark Holland). Detection was performed with a Quattro® Premier tandem mass spectrometer equipped with a Z Spray® ion source operated in positive electrospray ionisation mode (Waters). All aspects of system operation and data acquisition were controlled using MassLynx v4.1 software with automated data processing using the QuanLynx Application Manager (Waters).

On-line-SPE. On-line SPE was performed following a similar method as described by Kema et al. (17). The Symbiosis® system was designed to proceed automatically through a series of programmable routines during which the SPE cartridge was loaded, washed and eluted. The analytes were eluted directly on the analytical column and specified in Table 4.

TABLE 4
Table 4: Summary of the sequence of events used for on-line SPE.

| Step | Action | Details | Comment |
|---|---|---|---|
| 01 | New cartridge | Left clamp | Put cartridge in left clamp |
| 02 | Conditioning | Acetonitrile (1 mL at 2 mL/min) | Condition cartridge with acetonitrile |
| 03 | Equilibration | Water (2 mL at 2 mL/min) | Equilibrate cartridge with water |
| 04 | Start autosampler | Start loopfill | Fill loop autosampler with sample |
| 05 | Sample extraction | Water (500 μL at 500 μL/min) | Load sample on cartridge using water |
| 06 | Wash cartridge | Water (1 mL at 2.5 mL/min) | Wash cartridge with water |
| 07 | Wash cartridge | Acetonitrile:Water (9:1) | Wash cartridge with mixture of |
| | | (1 mL at 2.5 mL/min) | acetonitrile and water (9:1) |
| 08 | Transfer cartridge | Right clamp | Transfer cartridge from left to right clamp |
| 09 | Elution | LC gradient elution for 2.0 | Elution of analytes from cartridge directly |
| | | minutes | to the analytical column |
| 10 | New cartridge | Left clamp | Put cartridge in left clamp |
| 11 | Clamp flush | Right clamp | Wash cartridge and right clamp with 10 mmol/L |
| | | 10 mmol/L ammoniumformate | ammoniumformate buffer pH 3.0 |
| | | buffer pH 3.0 | |
| | | (1 mL at 1 mL/min) | |
| 12 | Clamp flush | Water (2 mL at 1 mL/min) | Wash cartridge and right clamp with |
| | | | water |
| 13 | Clamp flush | Acetonitrile (2 mL at 2 mL/min) | Wash cartridge and right clamp with |
| | | | acetonitrile |
| 14 | Move cartridge | Right clamp | Cartridge back to tray |

In the first step, the SPE cartridge was automatically located in the left clamp for conditioning and equilibration. Following this, the sample was passed on to the extraction cartridge using water as the loading solvent and wash solvents were applied. The extraction cartridge was then automatically transferred to the right clamp for elution of the analytes directly on the analytical column by passing the chromatographic mobile phase through the cartridge for 2 minutes. After elution, chromatographic separation on the analytical column occurred and the right clamp, containing the cartridge, was flushed. Processing of subsequent plasma samples was carried out in parallel.

Liquid chromatography. The binary gradient system consisted of 100 mmol/L ammonium-formate in water adjusted to pH 3.0 with formic acid (eluent A) and acetonitrile (eluent B). Gradient elution was performed according to the following elution program: 0-6 min, 5% A, 95% B; 6-7 min 20% A, 80% B; 7-8 min 20% A, 80% B; 8-9 min 5% A, 95% B; re-equilibration from 9-10 min with 5% A, 95% B. Gradients applied were linear; flow rate was 0.400 ml/min. Column temperature was kept at 20° C.

Mass spectrometry. In positive ionization mode, MN, NMN and 3-MT were protonated to produce ions at the form [M+H]⁺. These ions are known to undergo a facile loss of water in the ion source. Source conditions were optimised for these resulting ions of the form [M+H—H₂O]⁺: MN: m/z 180; NMN: m/z 166 and 3-MT: m/z 151). Upon collision induced dissociation (CID), these precursor ions produced characteristic product ions of m/z 148, m/z 134 and m/z 91, respectively. A multiple reaction monitoring (MRM) method was developed using a dwell time of 40 ms and an inter-channel delay of 10 ms.

### Quality Control and Method Validation

Selectivity. The identities of sample MN, NMN and 3-MT peaks were verified by analysis of the compound specific mass spectra after addition of calibrator (standard addition).

Detection limits. For plasma, detection limits (LOD) and quantification limits (LOQ) were determined by injecting serially diluted samples containing MN, NMN and 3-MT. LOD was defined as the injected amount that produced a signal-to-noise ratio of 3. LOQ was defined as the injected amount that produced a signal-to-noise ratio of 10. The percentage of carry-over between sequential analyses performed on new SPE cartridges was estimated by alternating injections of blanks and plasma samples with high concentrations of metanephrines.

Linearity and precision. The ratios of analyte peak area to internal standard peak area were plotted against metanephrines at eight concentrations in the range 0.26-18.21 nmol/L for MN, 0.82-18.21 nmol/L for NMN and 0.58-19.93 nmol/L for 3-MT. On seven different days fresh calibration lines were prepared and measured. The lines were calculated by Excel software using least-squares linear regression. The Clinical and Laboratory Standards Institute (CLSI; formerly NCCLS) EP-6P protocol was applied to test the linearity of the method. The dilutional linearity of the assay was performed in duplicate by serial dilution of spiked plasma samples with water. Intra- and inter-assay variation was determined by the use of three pooled samples with metanephrines in low, medium and high concentration (respectively 0.26, 1.55 and 11.57 nmol/L for MN; 0.82, 1.82 and 21.31 nmol/L for NMN and 0.58, 1.41 and 6.16 nmol/L for 3-MT). Intra-assay precision was obtained from 6 replicates measured in a single series. Inter-assay precision was assessed on 6 different days over a 3-week period.

Recovery. Mean relative recoveries were estimated by the addition of the metanephrines to plasma in low, medium and high concentrations. Recoveries were measured in 6 replicates of these samples by using two cartridges placed in series.

Stability. Spiked metanephrines samples (low, medium and high) were measured in triplicate after different storage conditions. The first set was assayed immediately and served as reference point; other sets were stored at 10° C. (autosampler temperature) and 4° C. for 16 h, 24 h, 48 h and 7 days and at room temperature for 24 h. The remaining samples were frozen at −20° C. and stability was investigated after 1-3 freeze-thaw cycles.

Biological variation, reference values and patient samples. Biological intra-day and inter-day variation were determined by analysing plasma obtained from 10 healthy persons in sitting position (5 males, 5 females, age range 20-56 years, median age 35.0), at 5 moments during one day and at 5 consecutive days, respectively. Metanephrines reference intervals were based on the analysis of 120 plasma samples derived from healthy controls in sitting position (63 males, 57 females, age range 36-81 years, median age 54.5), during the PREVEND study (19;20). Both studies were approved by the medical ethics committee of our institution and conducted in accordance with the guidelines of the Declaration of Helsinki. All participants gave written informed consent. Reference intervals were calculated using EP Evaluator (21) recommended by the CLSI.

Plasma samples from 10 patients with histologically proven pheochromocytoma were analysed to illustrate diagnostic value of the method.

### Results

### Quality Control and Method Validation

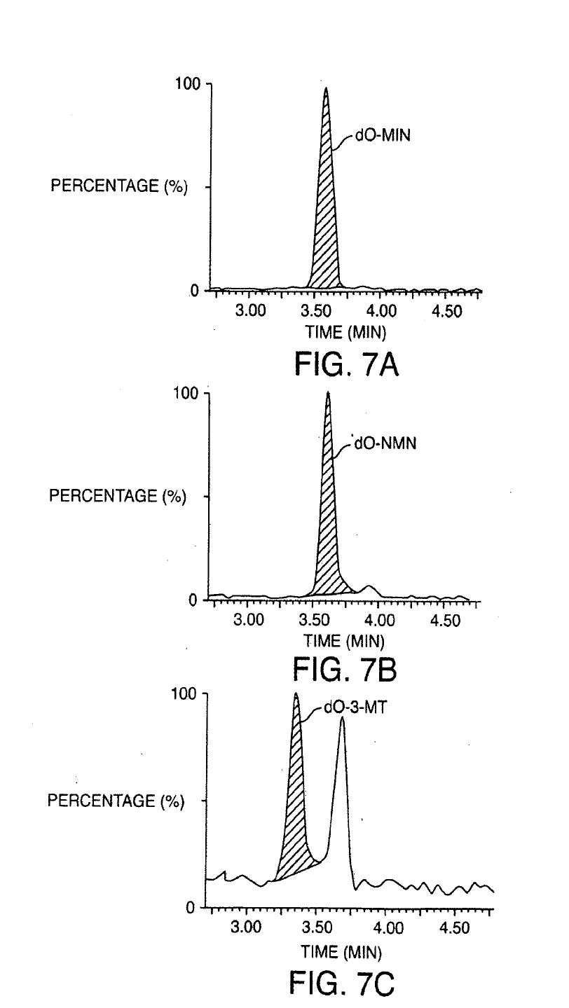

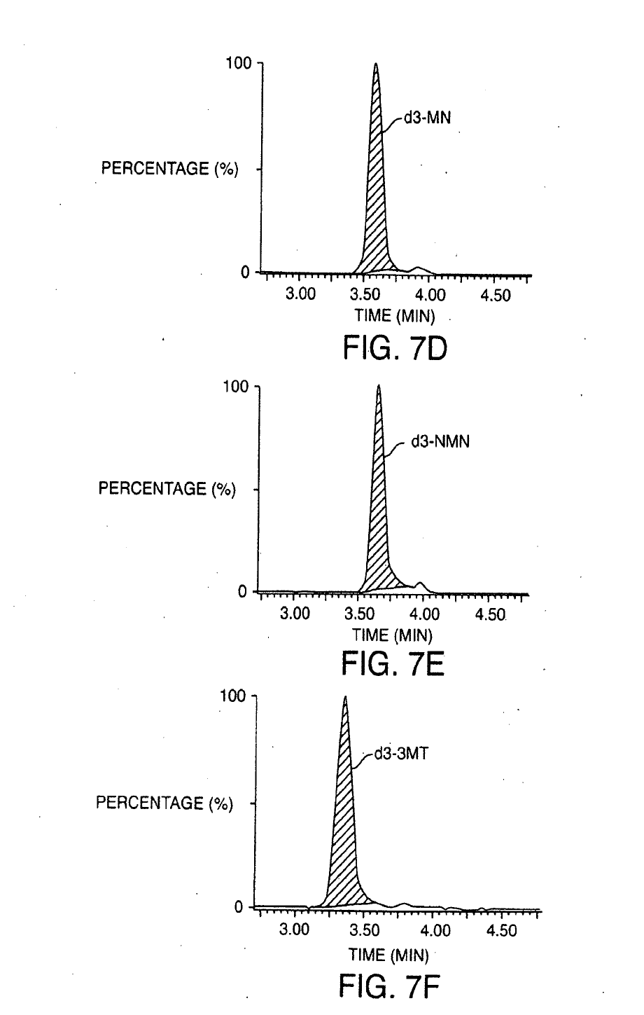

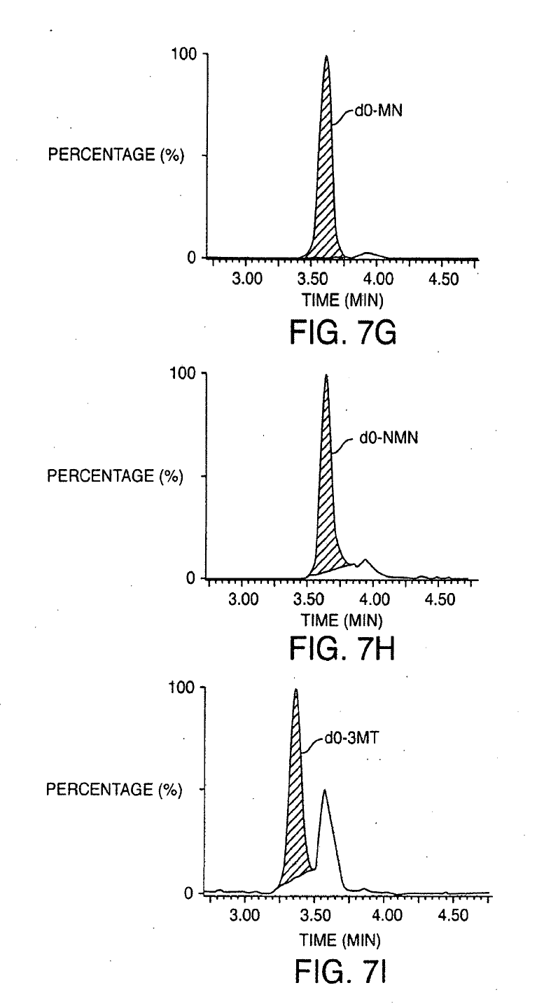

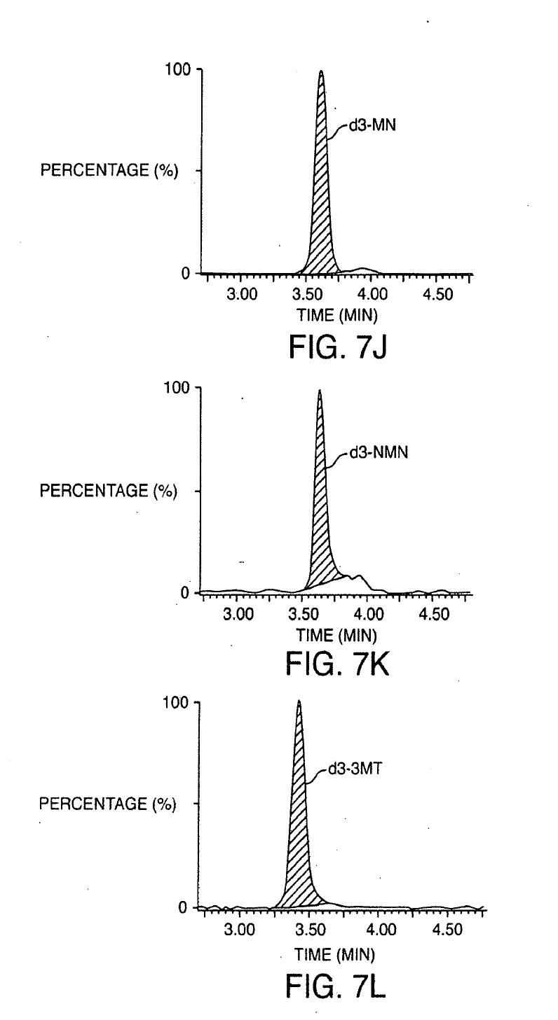

Chromatography and selectivity. Total sample analysis time, including extraction, is 11 min. Complete and time-consuming chromatographic separation of the metanephrines is not necessary and deuterated internal standards can be used, since the mass spectrometer monitors parent as well as daughter ions with high analytical specificity. Chromatograms obtained by XLC-MS/MS in MRM are shown in FIG. 7. The identities of the compounds were confirmed by the specific mass spectra. The masschromatograms in FIG. 7A-C show the responses for MN, NMN and 3-MT and the respective deuterated internal standards in a healthy control, while FIG. 7D-F show chromatograms with elevated responses from a patient with histologically proven pheochromocytoma. Not all pheochromocytoma patients have increased plasma free 3-MT levels. Comparison of the chromatograms from the healthy control with those of the pheochromocytoma patient reveals markedly increased MN (peak areas 3609 and 17034, respectively) and NMN concentrations (peak areas 1082 and 99150) in the patient sample. In healthy controls both MN and NMN are present in low but quantifiable amounts.

Detection limits. LOD was 0.05 nmol/L for MN, 0.05 nmol/L for NMN and 0.07 nmol/L for 3-MT. Quantification limits (at a signal-to-noise ratio of 10) were 0.10, 0.10 and 0.15 nmol/L, respectively.

Cartridges could be reused several times, without carry-over observed between sequential analyses performed on new or reused SPE cartridges, by applying additional washing steps to the method.

Linearity and precision. Plasma calibration curves and control samples were run with every batch of patient samples. Linearity was excellent over the 0-20 nmol/L calibration range with corresponding correlation coefficients (R²) consistently >0.99 for all three compounds. Plasma calibration curves were reproducible between days with R²>0.99. Mean analytical intra- and inter-assay repeatability and reproducibility for spiked pooled plasma in low, medium and high concentrations are shown in Table 5.

TABLE 5
Intra- and inter-assay precision of the XLC-MS/MS method for plasma free
MN, NMN and 3-MT.

| | Mean analytical variation (n = 6) | | | | | | Mean biological variation (n = 10) | | | | | |
|---|---|---|---|---|---|---|---|---|---|---|---|---|
| | Intra-assay | | | Inter-assay | | | Intra-day | | | Inter-day | | |
| | Mean | SD | CV | Mean | SD | CV | Mean | SD | CV | Mean | SD | CV |
| | nmol/L | nmol/L | % | nmol/L | nmol/L | % | nmol/L | nmol/L | % | nmol/L | nmol/L | % |
| MN | | | | | | | | | | | | |
| Low | 0.26 | 0.01 | 2.61 | 0.26 | 0.02 | 9.35 | 0.22 | 0.02 | 9.43 | 0.26 | 0.02 | 8.41 |
| Med | 1.55 | 0.03 | 1.97 | 1.55 | 0.04 | 2.28 | | | | | | |
| High | 11.57 | 2.08 | 2.85 | 11.57 | 0.18 | 1.56 | | | | | | |
| NMN | | | | | | | | | | | | |
| Low | 0.82 | 0.04 | 4.50 | 0.82 | 0.07 | 8.93 | 0.47 | 0.07 | 15.18 | 0.50 | 0.07 | 13.44 |
| Med | 1.82 | 0.04 | 2.01 | 1.82 | 0.04 | 2.39 | | | | | | |
| High | 21.24 | 3.96 | 4.74 | 21.24 | 0.95 | 4.46 | | | | | | |
| 3-MT | | | | | | | | | | | | |
| Low | 0.58 | 0.02 | 3.67 | 0.58 | 0.08 | 13.49 | 0.07 | 0.03 | 44.95 | 0.04 | 0.01 | 23.24 |
| Med | 1.41 | 0.04 | 2.95 | 1.41 | 0.07 | 4.72 | | | | | | |
| High | 6.16 | 1.06 | 2.12 | 6.16 | 0.22 | 3.53 | | | | | | |

Intra-assay CV (n=6) was 2.0-2.9% (MN), 2.0-4.7% (NMN) and 2.1-3.7% (3-MT). Inter-assay CV (n=6) was 1.6-9.4% (MN), 2.4-8.9% (NMN) and 3.5-13.5% (3-MT). Reproducibility, accuracy and precision were measured in the same way with aqueous calibration curves, which gave comparable results (data not shown). Plasma samples with high metanephrines concentrations, which exceed the calibration range, can be diluted up to 100 times.

Recovery. Recoveries ranged from 94.4 to 99.6% (MN), from 74.5 to 99.1% (NMN) and from 81.4 to 98.5% (3-MT) and are shown in Table 6 for low, medium and high concentration levels.

TABLE 6
Table 6. Mean recoveries of the XLC-MS/MS method for plasma free
MN, NMN and 3-MT.

| | Recovery analyte | Recovery IS |
|---|---|---|
| Compound | % (range) | % (range) |
| MN | | |
| Low | 98.9 (97.3-99.6) | 99.0 (98.3-99.4) |
| Med | 97.2 (94.4-99.5) | 97.2 (95.9-98.8) |
| High | 97.3 (95.8-98.6) | 97.3 (96.1-98.6) |
| NMN | | |
| Low | 94.6 (90.7-99.1) | 96.1 (93.7-98.3) |
| Med | 83.5 (76.7-95.2) | 83.1 (77.8-94.6) |
| High | 81.6 (74.5-93.7) | 82.0 (75.0-89.8) |
| 3-MT | | |
| Low | 91.1 (85.2-96.0) | 96.6 (91.3-99.0) |
| Med | 90.4 (81.4-96.6) | 94.6 (92.2-98.4) |
| High | 96.6 (92.8-98.5) | 96.7 (89.9-98.0) |

Stability. Metanephrines were stable in human plasma stored up to 7 days at 10° C. or 4° C. At room temperature plasma metanephrines were stable up to 24 h. No changes in measured concentrations were observed in plasma that had been subjected to one, two or three freeze-thaw cycles. Stability data (n=3) are not shown.

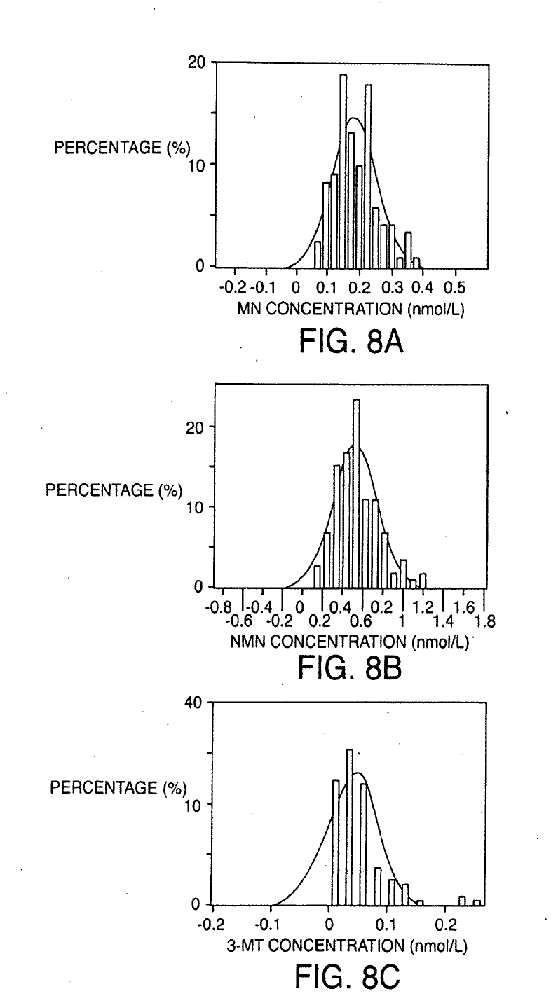

Biological variation, reference values and patient samples. Biological intra- and inter-day CV (n=10) were 9.4 and 8.4% (MN), 15.2% and 13.4% (NMN) and 44.9% and 23.2% (3-MT), respectively, as is shown in Table 2. The distribution of 120 reference values is right shifted for all three metanephrines, as is shown in FIGS. 8a, 8b and 8c. Therefore reference intervals were calculated with EP evaluator in a transformed parametric manner according to the CLSI C28-A2 (22). Reference intervals were 0.07-0.33 nmol/L (MN), 0.23-1.07 nmol/L (NMN) and <0.17 nmol/L (3-MT). Elevated plasma free NMN and/or MN concentrations of 10 patients with histologically proven pheochromocytoma, measured with XLC-MS/MS, ranged from 0.11-17.93 (mean 7.26) nmol/L (MN) and from 4.05-70.10 (mean 25.84) nmol/L (NMN).

Thus, we have described methods and apparatus for the measurement of plasma free metanephrines. The methods and apparatus are accurate, precise and linear with high throughput. It offers advantages over off-line methods with regard to analytical variation and sample preparation time and costs, while it allows measurement of large samples series due to short analysis time. Furthermore, the method has high sensitivity and specificity by the use of XLC-MS/MS and measures in the nanomolar range.

We have described preferred embodiments of the present invention in detail with the understanding that such embodiments are capable of modification and alteration without departing from the teaching herein. Therefore, the invention should not be limited to the precise details but should encompass the subject matter of the claims that follow and their equivalents.

1. A method of detecting at least one of the group of compounds selected from normetanephrine, metanephrine and 3-methoxytyramine in blood plasma, the presence of which is potentially indicative of catecholamine secreting tumors, comprising the steps of:
a. obtaining a plasma sample from a subject;
b. separating at least one of normetanephrine, metanephrine and 3-methoxytyramines from plasma proteins and constituents with a weak cationic exchange resin to form at least one retained normetanephrine, metanephrine or 3-methoxytyramine;
c. eluting at least one retained normetanephrine, retained metanephrine and retained 3-methoxytyramine from the weak cationic exchange resin with a acid mobile phase to form a at least one normetanephrine eluant, metanephrine eluant and 3-methoxytyramine eluant eluant;
d. separating the at least one normetanephrine eluant, metanephrine eluant and 3-methoxytyramine eluant into at least one fine normetanephrine eluant, fine metanephrine eluant and fine 3-methoxytyramine eluant through on line hydrophilic interaction liquid chromatography, said fine normetanephrine eluant, fine metanephrine eluant and fine 3-methoxytyramine eluant exhibiting one or more peaks corresponding to the presence of at least one of group consisting of metanephrine and normetanephrine;
e. introducing said fine normetanphrine and metanephrine eluant into a mass spectrometer to produce a signal relating to the presence of and quantity of at least one of said group consisting of normetanephrine, metanephrine and 3-methoxytyramine, said presence and quantity is indicative of the presence or absence of tumors secreting catecholamines.

2. The method of claim 1 further comprising the step of adding a internal standard to said sample.

3. The method of claim 2 wherein said internal standard is at least one known quantity of a deuterated form of the compound selected from the group consisting of normetanephrine, metanephrine and 3-methoxytyramine.

4. The method of claim 1 wherein said weak cationic exchange resin is a water-wettable organic polymer with one or more weak cationic exchange functional groups.

5. The method of claim 4 wherein said organic polymer is derived from the monomers selected from at least one hydrophilic monomer and at least one hydrophobic monomer.

6. The method of claim 5 wherein said hydrophobic monomer is divinylbenzene.

7. The method of claim 5 wherein said hydrophilic monomer is selected from the pair of monomers consisting of N-vinylpyrrolidone and vinylpyridine.

8. The method of claim 4 wherein said weak cationic exchange group is carboxyl.

9. The method of claim 3 wherein said plasma levels of catecholamines is determined by comparing unlabeled normetanephrine levels to deuterated normetanephrine concentration in said sample.

10. The method of claim 3 wherein said plasma levels of catecholamines is determined by comparing unlabeled metanephrine levels to deuterated metanephrine concentration in said sample.

11. The method of claim 1 wherein said mass spectrometer is operated in positive ionization mode.

12. The method of claim 11 wherein metanephrine is protonated to form an ion of mass to charge 198.

13. The method of claim 11 wherein said normetanephrine is protonated to form an ion of mass to charge 184.

14. The method of claim 11 wherein 3-methoxytyramine is protonated to form an ion of mass to charge ratio (m/z) 168.

15. The method of claim 12 wherein said metanephrine loses water to form an ion of having a mass to charge of 180.

16. The method of claim 13 wherein said normetanephrine loses water to form an ion of having a mass to charge of 166.

17. The method of claim 14 wherein said 3-methoxytyramine will typically lose ammonia to form an ion having a mass to charge ratio of 151.

18. The method of claim 1 wherein said mass spectrometer employs collisional fragmentation to form one or more ion fragments of metanephrine.

19. The method of claim 18 wherein said fragments of metanephrine has a mass to charge of 148.

20. The method of claim 1 wherein said mass spectrometer employs collisional fragmentation to form one or more ion fragments of normetanephrine.

21. The method of claim 20 wherein said fragments of normetanephrine has a mass to charge of 134.

22. The method of claim 1 wherein said mass spectrometer employs collisional fragmentation to form one or more ion fragments of 3-methoxytyramine.

23. The method of claim 22 wherein said fragments of 3-methoxytyramine having a mass to charge ratio of 119.

24. The method of claim 1 wherein the concentration of metanephrine greater than about 0.3 nmole per liter suggests pheochromocytoma.

25. The method of claim 1 wherein the concentration of normetanephrine greater than about 1.2 nmole per liter suggest pheochromocytoma.

26. The method of claim 1 wherein the concentration of of 3-methoxytyramine greater than about 0.2 nmole per liter suggests pheochromocytoma.

27. The method of claim 3 wherein said plasma levels of catecholamine is determined by comparing unlabeled 3-methoxytyramine levels to deuterated 3-methoxytyramines concentration in said sample.

28. An apparatus for detecting at least one of the group of compounds selected from normetanephrine, metanephrine and 3-methoxytyramine in blood plasma, the presence of which is potentially indicative of catecholamine secreting tumors, comprising:
a. separating and eluting means for separating at least one of the group of compounds selected from normetanephrine, metanephrine and 3-methoxytyramines from plasma proteins and constituents with a weak cationic exchange resin to form at least one of a retained normetanephrine, retained metanephrine and retained 3-methoxytyramine, and eluting the at least one of the retained normetanephrine, retained metanephrine and retained 3-methoxytyramine from the weak cationic exchange resin with a acid mobile phase to form at least one of a normetanephrine eluant, metanephrine eluant and 3-methoxytyramine eluant, said separating and eluting means receiving one or more plasma samples from one or more subjects, and in fluid communication with liquid chromatograph means and in signal communication with control means said separating and eluting means sending a sample identifier signal to said control means;
b. liquid chromatograph means for separating the at least one of normetanephrine eluant, metanephrine eluant and 3-methoxytyramine eluant into at least one fine normetanephrine eluant, fine metanephrine eluant and fine 3-methoxytyramine eluant through on line hydrophilic interaction liquid chromatography, said at least one fine normetanephrine eluant, fine metanephrine eluant and fine 3-methoxytyramine eluant exhibiting one or more peaks corresponding to the presence of at least one of group consisting of metanephrine, normetanephrine and 3-methoxytyramine, said liquid chromatograph means in fluid communication with mass spectrometer means and signal communication with control means, said liquid chromatograph maintaining said at least one fine normetanephrine eluant, fine metanephrine eluant and fine 3-methoxytyramine eluant in association with said sample identifier signal;
c. mass spectrometer means for receiving said at least one fine normetanphrine eluant, fine metanephrine eluant and fine 3-methoxytyramine eluant to produce at least one mass to charge signal relating to the presence of and quantity of at least one of said group consisting of normetanephrine, metanephrine and 3-methoxytyramine, said mass spectrometer means in signal communication with control means;
d. control means for receiving said sample identifier signal and mass to charge signal and associating said signal to a mass to charge signal said sample identifier signal and comparing said mass to charge signal to control values to suggest the presence or absence of tumors secreting catecholamines to a subject.

29. A kit for detecting at least one of the group of compounds selected from normetanephrine, metanephrine and 3-methoxytyramine in blood plasma, the presence of which is potentially indicative of catecholamine secreting tumors, comprising at least one of the compounds normetanephrine, metanephrine and 3-methoxytyramine and instructions for forming standards for adding to plasma samples and for calibrating mass spectrometers.
